(19) United States
(12) Patent Application Publication
(10) Pub. No.: US 2022/0202620 A1
(43) Pub. Date: Jun. 30, 2022

(54) Manifold With Biological Actives For Negative-Pressure Therapy

(71) Applicant: KCI Licensing, Inc., San Antonio, TX (US)

(72) Inventors: Diwi L. ALLEN, San Antonio, TX (US); Prathamesh Madhav KHARKAR, San Antonio, TX (US)

(21) Appl. No.: 17/605,168

(22) PCT Filed: Mar. 20, 2020
(86) PCT No.: PCT/US2020/023880

§ 371 Date: Oct. 20, 2021

Related U.S. Application Data
(60) Provisional application No. 62/845,174, filed on May 8, 2019.

(51) Int. Cl. A61F 13/00; A61M 1/00; A61M 35/00; A61K 38/39; A61K 31/727; A61K 31/737

(52) U.S. Cl. CPC A61F 13/00063; A61M 1/90; A61F 13/00068; A61M 35/10; A61M 2207/00; A61K 31/727; A61K 31/737; A61M 2202/064; A61K 38/39

(57) ABSTRACT

A system for use in treating a tissue site with negative pressure, which may comprise a dressing or tissue interface and a plurality of standoffs for storing and releasing a biocompatible polymer to the tissue site. The standoff may be cells or closed-end cells. In some examples, the biocompatible polymer may comprise collagen, oxidized regenerated cellulose, or a combination thereof. Method for using and manufacturing the dressing or tissue interface may also be disclosed.

### RELATED APPLICATION

The present invention claims the benefit of the filing of U.S. Provisional Patent Application No. 62/845,174, filed May 8, 2019, which is incorporated herein by reference for all purposes.

### TECHNICAL FIELD

The invention set forth in the appended claims relates generally to tissue treatment systems and more particularly, but without limitation, to dressings for tissue treatment with negative-pressure and methods of manufacturing and using the dressings for tissue treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a system for treating a tissue site may comprise a tissue interface comprising a plurality of standoffs configured for storing and releasing a biocompatible polymer to the tissue site. The system may further comprise a cover configured to be disposed adjacent to the tissue interface and to form a seal around the tissue site. The system may also comprise a negative-pressure source fluidly coupled to the tissue interface through the cover. The tissue interface may comprise thermoplastic elastomers, polyurethane, polyethylene, silicone, silicone-like materials, polyamide, polypropylene, polyethylene, polyvinyl chloride, ethylene vinyl acetate copolymers, polyvinyl alcohol, polyether block amide (PEBAX) polymers, or any combination thereof.

The biocompatible polymer may aid wound healing; for example, the polymer may have anti-inflammatory, matrix metalloproteinase (MMP)-mitigating, antimicrobial properties, or a combination thereof. The biocompatible polymer may be biosorbable. The biocompatible polymer may have a biological origin or synthetic or comprise both biological polymers and synthetic polymers. For example, the polymer may comprise collage, oxidized cellulose such as oxidized regenerated cellulose, or a combination thereof. The term “oxidized cellulose” refers to any material produced by the oxidation of cellulose, for example with dinitrogen tetroxide. Such oxidation converts primary alcohol groups on the saccharide residues to carboxylic acid groups, forming uronic acid residues within the cellulose chain. The oxidation generally does not proceed with complete selectivity, and as a result hydroxyl groups on carbons 2 and 3 are occasionally converted to the keto form. These keto units introduce an alkali-labile link, which at pH 7 or higher initiates the decomposition of the polymer via formation of a lactone and sugar ring cleavage. As a result, oxidized cellulose may be biodegradable and resorbable or bioresorbable under physiological conditions. In some embodiments, the polymer may comprise oxidized regenerated cellulose (ORC), which may be prepared by oxidation of a regenerated cellulose, such as rayon.

Additionally or alternatively, the biocompatible polymer may comprise hyaluronic acid, chitosan, heparin, alginate, cellulose, fibrin, gelatin, chondroitin sulfate, agarose, dextran, carrageenan, silk, poly(ethylene glycol), poly(vinyl alcohol), polycaprolactone, polyphophazene, polyglycolic acid, rosin, lactose, sucrose, tapioca starch, and polyvinylpyrrolidone, or any combination thereof. For example, the biocompatible polymer may comprise inert polymers such as poly(ethylene glycol), poly(vinyl alcohol), polycaprolactone, polyphophazene, polyglycolic acid, and polyvinylpyrrolidone.

In more specific examples, the standoffs may comprise cells, blisters, bubbles, or other raised formations. The standoffs may have a width of between 0.5 mm and 10 mm and may have a height of 0.5 mm and 10 mm. In some examples, the standoffs may have perforations or slits to permit fluid exchange.

Alternatively, other example embodiments may comprise a dressing comprising a manifold comprising a film and a first side and a second side. The manifold may comprise a plurality of standoffs, wherein at least some of the standoffs may enclose a composition comprising a biocompatible polymer. The dressing may further comprise a cover configured to be adjacent to the second side of the manifold and to form a sealed therapeutic environment between a tissue site and the first side of the manifold. A manifold comprising a polymer film and a plurality of standoffs enclosing a composition comprising a biocompatible polymer is also described herein.

Alternatively, in another example embodiment, a method for treating a tissue site is disclosed comprising positioning a manifold adjacent to the tissue. The manifold may comprise a polymeric film having a plurality of standoffs, or more particularly, cells enclosing a compositing comprising a biocompatible polymer. The method may further comprise covering the manifold and the issue site with a cover to form a sealed therapeutic environment. The method may further comprising providing negative pressure from a negative-pressure source to the tissue site through the manifold. In some embodiments, the cells do not block transmission of negative pressure to the tissue site.

In another example embodiment, a method for manufacturing a manifold may be provided as comprising providing a polymeric film having a plurality of cells. The method may further comprise incorporating a composition comprising a biocompatible polymer into at least some of the cells. The method may further comprise sealing the cells to encapsulate the composition.

In another example embodiment, a method for manufacturing a manifold may be provided as comprising providing a polymeric film and wrapping at least some portion of the polymeric film around a composition comprising a biocompatible polymer. The method may further comprising sealing the portions of the polymer film to create cells to encapsulate the composition.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a graph illustrating additional details of example pressure control modes that may be associated with some embodiments of the therapy system of FIG. 1;

FIG. 3 is a graph illustrating additional details that may be associated with another example pressure control mode in some embodiments of the therapy system of FIG. 1;

FIG. 4 is a chart illustrating details that may be associated with an example method of operating the therapy system of FIG. 1;

FIG. 5A is a schematic view of additional details that may be associated with various examples of features of a tissue interface;

FIG. 5B is a schematic view of features of FIG. 5A taken along section 5B-5B, illustrating additional details that may be associated with some examples;

FIG. 5C is a schematic view of alternative features of FIG. 5A taken along section 5B-5B, illustrating additional details that may be associated with some examples; and

FIG. 6 is a schematic view illustrating additional details that may be associated with an alternative example embodiment of therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

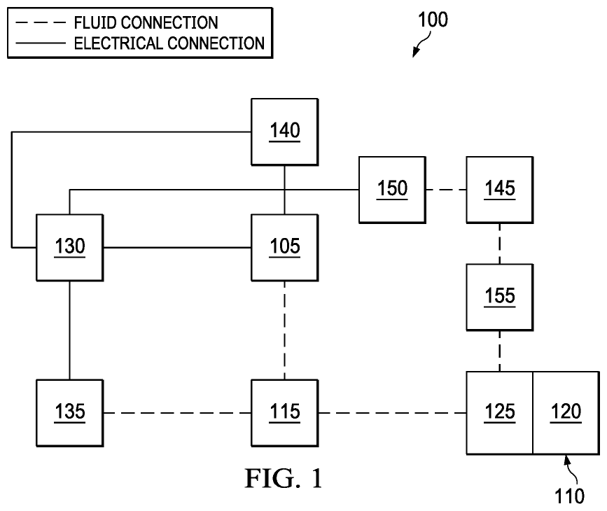

FIG. 1 is a simplified functional block diagram of an example embodiment of a therapy system 100 that can provide negative-pressure therapy with instillation solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source, such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. In some embodiments, a manifold may additionally or alternatively comprise projections that form interconnected fluid pathways.

The thickness of the tissue interface 120 may also vary according to needs of a prescribed therapy. For example, the thickness of the tissue interface 120 may be decreased to reduce tension on peripheral tissue. The thickness of the tissue interface 120 can also affect the conformability of the tissue interface 120. In some embodiments, a thickness in a range of about 0.5 millimeters to 5 centimeters may be suitable, preferably in the range of 0.5 millimeters to 1 centimeter.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes may have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, California; polyether block polyamide copolymer (PEBAX), for example, from Arkema S. A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

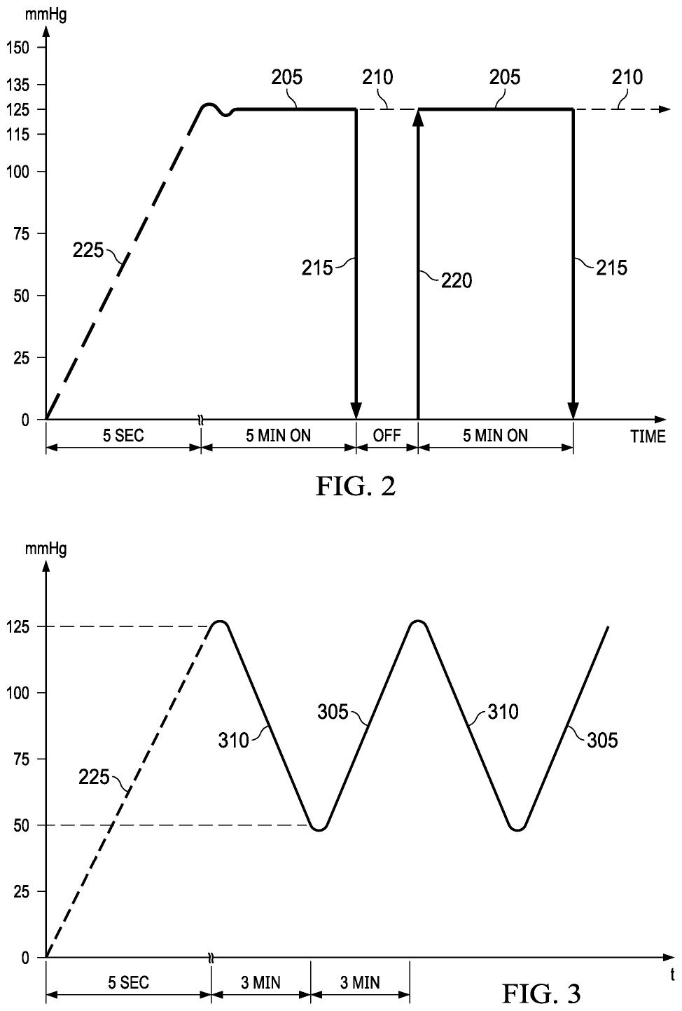

FIG. 2 is a graph illustrating additional details of an example control mode that may be associated with some embodiments of the controller 130. In some embodiments, the controller 130 may have a continuous pressure mode, in which the negative-pressure source 105 is operated to provide a constant target negative pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time and the y-axis represents negative pressure generated by the negative-pressure source 105 over time. In the example of FIG. 2, the controller 130 can operate the negative-pressure source 105 to cycle between a target pressure and atmospheric pressure. For example, the target pressure may be set at a value of 135 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time, as indicated by the solid line 220, may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a negative pressure of 50 and 135 mmHg with a rise time 305 set at a rate of +25 mmHg/min. and a descent time 310 set at −25 mmHg/min. In other embodiments of the therapy system 100, the triangular waveform may vary between negative pressure of 25 and 135 mmHg with a rise time 305 set at a rate of +30 mmHg/min and a descent time 310 set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

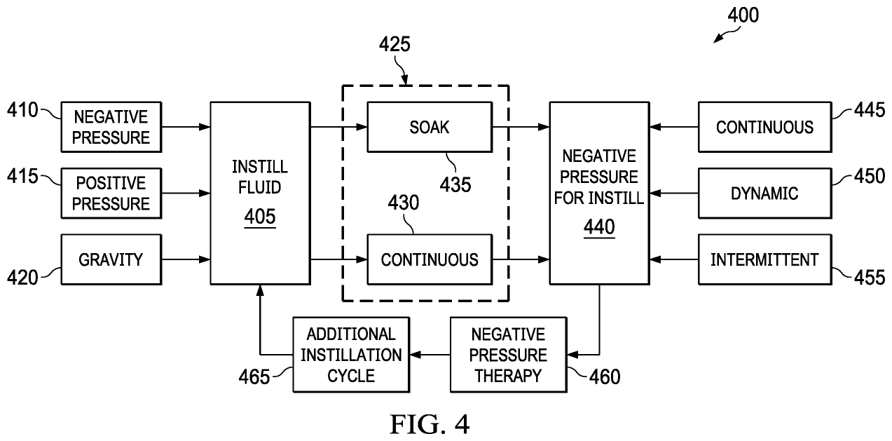

FIG. 4 is a chart illustrating details that may be associated with an example method 400 of operating the therapy system 100 to provide negative-pressure treatment and instillation treatment to the tissue interface 120. In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution, as indicated at 405. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120, as indicated at 410. In some embodiments, solution may be instilled to a tissue site by applying a positive pressure from the positive-pressure source 160 to move solution from the solution source 145 to the tissue interface 120, as indicated at 415. Additionally or alternatively, the solution source 145 may be elevated to a height sufficient to allow gravity to move solution into the tissue interface 120, as indicated at 420.

The controller 130 may also control the fluid dynamics of instillation at 425 by providing a continuous flow of solution at 430 or an intermittent flow of solution at 435. Negative pressure may be applied to provide either continuous flow or intermittent flow of solution at 440. The application of negative pressure may be implemented to provide a continuous pressure mode of operation at 445 to achieve a continuous flow rate of instillation solution through the tissue interface 120, or it may be implemented to provide a dynamic pressure mode of operation at 450 to vary the flow rate of instillation solution through the tissue interface 120. Alternatively, the application of negative pressure may be implemented to provide an intermittent mode of operation at 455 to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied at 460. The controller 130 may be utilized to select a mode of operation and the duration of the negative pressure treatment before commencing another instillation cycle at 465 by instilling more solution at 405.

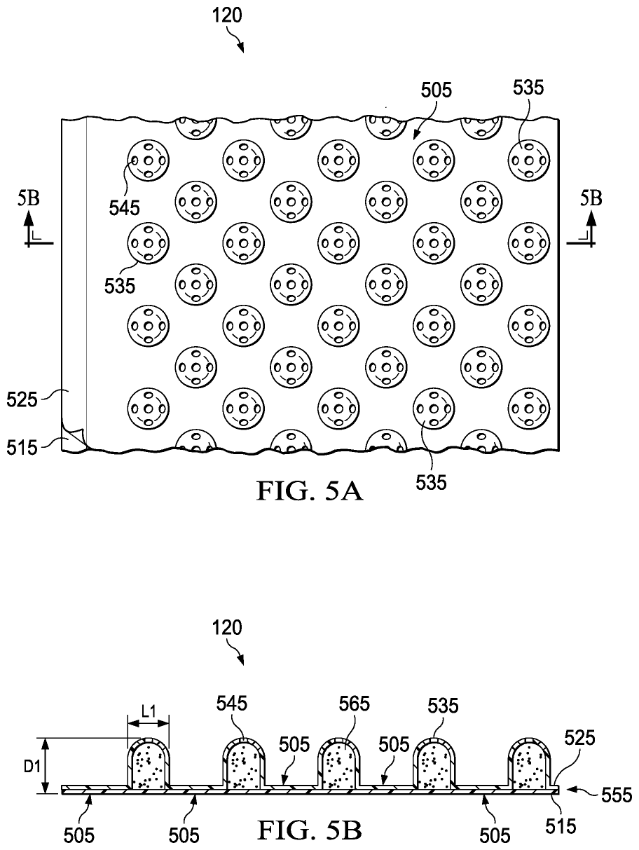

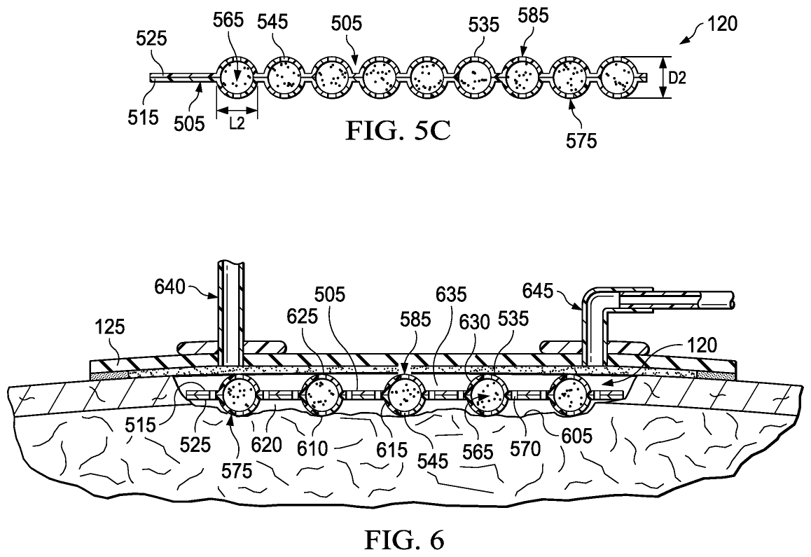

FIG. 5A is a schematic view of additional details that may be associated with various examples of the tissue interface 120. In the example of FIG. 5A, the tissue interface 120 may comprise one or more layers. For example, the tissue interface 120 may comprise a first layer 515 sealed to a second layer 525. In other embodiments, the tissue interface 120 may comprise a single layer. The tissue interface 120 may be pliable, preferably for tailoring to dimensions of a tissue site. To increase additional pliability, the tissue interface 120 may be soaked in an aqueous solution before application in a wound environment. For example, the aqueous solution may be phosphate-buffered saline (PBS).

In some embodiments, the tissue interface 120, or any of its layers, like the first layer 515 or the second layer 525, may have a thickness within a range of between 0.5 mm to 5 cm, or preferably, within a range of between 0.5 mm to 1 cm. In further embodiments, the dimensions of the tissue interface 120 may be varied to be tailored to dimensions of a tissue site.

In further embodiments, the tissue interface 120, or any of its layers, like the first layer 515 or the second layer 525, may be formed from a material comprising thermoplastic elastomers, polyurethane, polyethylene, silicone, silicone-like materials, polyamide, polypropylene, polyethylene, polyvinyl chloride, ethylene vinyl acetate copolymers, polyvinyl alcohol, polyether block amide (PEBAX) polymers, or any combination thereof. In some embodiments, the tissue interface 120 may be fabricated using a thermoplastic elastomer. Additionally, one or more of the layers of the tissue interface 120 may also have a smooth or matte surface texture in some embodiments. For example, the first layer 515 may have a smooth or matte surface texture in some embodiments.

In some embodiments, the tissue interface 120 may comprise a plurality of standoffs 535. In some embodiments, the standoffs 535 may comprise protrusions, blisters, bubbles, cells or other raised formations that extend above or below the tissue interface 120. In particular embodiments, the standoffs 535 may comprise bubbles or cells having a closed end.

The standoffs 535 in adjacent rows or columns may be staggered so that the standoffs 535 may be nested or packed together, as illustrated in the example of FIG. 5A. In other embodiments, the standoffs 535 may be arranged in other patterns suitable for the particular therapy being utilized. For example, the rows and columns of the standoffs 535 may be arranged in line to form an aligned, rectangular pattern so that there is more spacing between the standoffs 535.

In some embodiments, the standoffs 535 may extend from the second layer 525 on one side of the tissue interface 120 as illustrated in FIG. 5B. At least some of the standoffs 535 may be configured to come in direct contact with a tissue site. In other examples, the flat surface of the first layer 515 may be configured to contact a tissue site. The standoffs 535 may be flexible, semi-rigid, or rigid. The pattern and position of the standoffs 535 may be uniform or non-uniform. The standoffs 535 may have any suitable shapes, including but not limited to, for example, the shape of a tube, a hemisphere, a sphere, a circle, a polygon, a spike, a cone, a pyramid, a dome, a cylinder, a rectangle, any regular or irregular shape, or any combinations thereof. These shapes may be formed in one or both sheets of the tissue interface 120 such as the single hemispherical shape shown in standoffs 535 in FIG. 5B.

The standoffs 535 may be formed as an integral part of the tissue interface 120 or any of its layers and, therefore, they may also be formed from the same material as the tissue interface 120 or any of its layers. In one embodiment, the standoffs 535 may be formed from a substantially gas-impermeable material such as silicone. In another embodiment, the standoffs 535 may be formed from a semi-gas permeable material. The standoffs 535 may be configured not to block the transmission of negative pressure to a tissue site.

FIG. 5B is a schematic view of the features of FIG. 5A taken along section 5B-5B, illustrating additional details that may be associated with some examples. In some embodiments, the tissue interface 120 may comprise at least two sheets or films of a polymer. The films may have inner surfaces coupled to each other in a pattern defining a plurality of standoffs 535, separated by space regions 505, as illustrated in the example of FIG. 5B. In other example embodiments, the tissue interface 120 may comprise or consist essentially of a single sheet comprising standoffs 535. For example, the standoffs 535 may have a width L1 of from 0.5 mm to 10 mm. The standoffs 535 may have a length D1 of from 0.5 to 10 mm. The spacing between adjacent standoffs 535 may be within a range from 2.0 mm to 10 mm apart. The standoffs 535 may have a wall with a thickness between 0.5 mm to 4.5 cm.

In some embodiments, the standoffs 535 may have a hemispherical profile, as illustrated in the example of FIG. 5B. In other example embodiments, the standoffs 535 may have profiles that are conical, cylindrical, tubular having a flattened or hemispherical end, or geodesic. The standoffs 535 may be tubular in some embodiments, formed with generally parallel walls extending from the base 555 to a hemispherical or flat top portion of the standoffs 535. Alternatively, the walls of the standoffs 535 may taper or expand outwardly from the base 555. In some embodiments, the standoffs 535 that are generally hemispherical or tubular in shape may have a diameter between 0.5 mm and 10 mm. In some embodiments, space regions 505 between the standoffs 535 may have apertures or may have fluid-permeable portions so the negative pressure or instillation fluids can be distributed through the apertures or fluid-permeable portions to a tissue site. In other embodiments, space regions 505 may be airtight or fluid-impermeable to so the negative pressure or instillation fluids can be distributed to a tissue site through a periphery of the tissue interface 120.

As illustrated in FIGS. 5B, in certain embodiments, the standoffs 535 may contain, enclose, or encapsulate a polymer 565. The polymer 565 may be a biocompatible polymer. The polymer 565 may be a biologically-active polymer, such as an active ingredient that aids in wound healing. The polymer 565 may be a biosorbable polymer, a biologically-active polymer, or a biosorbable, biologically active polymer. The polymer 565 may be a biologically-active polymer, an inert polymer, or a combination thereof. The polymer 565 may be a synthetic polymer, a semi-synthetic polymer, a polymer with a biological origin, or a combination thereof. For example, the polymer 565 may comprise collagen, oxidized regenerated cellulose, hyaluronic acid, chitosan, heparin, alginate, cellulose, fibrin, gelatin, chondroitin sulfate, agarose, dextran, carrageenan, silk, poly(ethylene glycol), poly(vinyl alcohol), polycaprolactone, polyphophazene, polyglycolic acid, rosin, lactose, sucrose, tapioca starch, and polyvinylpyrrolidone, or any combination thereof. In one embodiments, the polymer 565 may comprise a combination of collagen and oxidized regenerated cellulose. In further embodiments, the polymer 565 may comprise cross-linked collagen polymers to slow down the degradation time in response to collagenase in the wound exudate.

The polymer 565 enclosed within the standoffs 535 can be released over a selected time period or a desired time period, for example, at least two days, three days, four days, five days, seven days, or 21 days or any intermediate time period. In certain embodiments, the polymer 565 may be released over a seven-day time period, depending on enzyme activity of wound exudate. To regulate or accelerate the release efficiency of the polymer 565 from the standoffs 535, the standoffs 535 may comprise apertures 545, such as slits, to allow or accelerate fluid flow of wound exudate from a tissue site and access of the polymer 565 to the tissue site. Additionally or alternatively, the apertures 545 may be disposed in the first layer 515 adjacent to the base of the standoffs 535. In some embodiments, the apertures 545 may be arranged in cross hatches. For example, the apertures 545 may have a diameter, width, or length of between 0.5 to 2 mm.

The size and spacing of the apertures 545 of the standoffs 535 also may be varied to effect change in the fluid flows through the apertures 545. For example, the diameter and spacing of the apertures 545 can be increased to increase the release rate of the polymer 565. The size, spacing, or both of the apertures 545 may be decreased to restrict fluid flow, which can slow down the release rate of the polymer 565.

The tissue interface 120 may be formed by incorporation of the polymer 565 into one or more of the standoffs 535. In some embodiments, the polymer 565 may be in a liquid form, such as a liquid slurry, and may be injected into the standoffs 535. Additionally or alternatively, the polymer 565 can be injected into the standoffs 535 by a needle. The size of the needle hole may be selected so that the polymer 565 cannot leak back out from the needle hole. In further embodiments, the polymer 565 encapsulated in the standoffs 535 may undergo a freeze-drying process before applying to a tissue site.

Additionally or alternatively, the polymer 565 can be in a solid form, a liquid form, or a mixture thereof. For example, the polymer 565 can be a powder. Additionally or alternatively, the instillation fluids may comprise an activating agent configured to activate the polymer 565 that were inactive or had a lower efficacy before activation.

Additionally or alternatively, one or more polymer films may be wrapped around the polymer 565, which may be in a solid form, and subsequently sealed to create standoffs 535 encapsulating the polymer 565. The polymer films may be sealed by heat or any other means, such as an adhesion bond.

Additionally or alternatively, the tissue interface 120 or any of its layers or the standoff 535 may provide a means for controlling or managing fluid flow. For example, the space regions 505 may have one or more fluid restrictions (not shown). The fluid restrictions may be bi-directional and pressure-responsive. For example, a fluid restriction generally may comprise or consist essentially of an elastic passage that is normally unstrained to substantially reduce liquid flow, and can expand or open in response to a pressure gradient. For example, in some embodiments, the fluid restrictions may comprise or consist essentially of fenestrations or perforations in the space regions 505. Some embodiments of the fluid restrictions may comprise or consist essentially of one or more slits, slots or combinations of slits and slots in the space regions 505. In some examples, the fluid restrictions may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable.

In some embodiments, the material used for the tissue interface 120 or any of its layers or the standoffs 535 may possess sufficient tensile strength to resist stretching under apposition forces created by negative-pressure therapy. The tensile strength of a material is the ability of material to resist stretching as represented by a stress-strain curve where stress is the force per unit area, i.e., pascals (Pa), newtons per square meter (N/m²), or pounds per square inch (psi). The ultimate tensile strength (UTS) is the maximum stress the material can withstand while being stretched before failing or breaking. Many materials display a linear elastic behavior defined by a linear stress-strain relationship often extending up to a nonlinear region represented by the yield point, i.e., the yield strength of a material. For example, high density polyethylene (HDPE) has a high tensile strength and low-density polyethylene (LDPE) has a slightly lower tensile strength, which are suitable materials for the sheets of non-porous, polymeric film as set forth above. Linear low density polyethylene (LLDPE) may also be suitable for some examples because the material stretches very little as the force is increased up to the yield point of the material. Thus, the standoffs 535 can be configured to resist collapsing (or stretching) when subjected to an external force or pressure. For example, HDPE has a UTS of about 37 MPa and may have a yield strength that ranges from about 26-33 MPa depending on the thickness of the material, while LDPE has somewhat lower values.

In some example embodiments, the material used for the tissue interface 120 or any of its layers or the standoffs 535 may comprise or consist essentially of a thermoplastic polyurethane (TPU) film that is permeable to water vapor but impermeable to liquid. The film may be in various degrees breathable and may have MVTRs that are proportional to their thickness. For example, the MVTR may be at least 300 g/m² per twenty-four hours in some embodiments. For permeable materials, the permeability generally should be low enough to maintain a desired negative pressure for the desired negative-pressure treatment.

In some example embodiments, the thermoplastic polyurethane film may be, for example, a Platilon® thermoplastic polyurethane film available from Convestro LLC, which may have a UTS of about 60 MPa and may have a yield strength of approximately 11 MPa or greater than about 10 MPa depending on the thickness of the material. Therefore, in some example embodiments, it is desirable that the non-porous, polymeric film may have a yield strength greater than about 10 MPa, depending on the type and thickness of material. A material having a lower yield strength may be too stretchable and, therefore, more susceptible to breaking with the application of small amounts of compression and/or apposition forces.

In some examples, the tissue interface 120 of FIG. 5A may be manufactured from a roll of polymer films with predefined dimensions. The tissue interface 120 may also comprise separation paths, which can allow the tissue interface 120 to be sized for a tissue site. The separation paths may be non-leaking so that the composite manifold may still be utilized when not torn into separate components. In some example embodiments, the separation paths may be formed by indentations in the polymeric film that provide a weakened path in the film to facilitate tearing by a caregiver. In other example embodiments, the tissue interface 120 may comprise two sheets of polymeric film, wherein the non-leaking tear paths may be formed by perforations or apertures in at least one of the two sheets of polymeric film. If perforations or apertures are formed in both of the two sheets of polymeric film to further facilitate tearing, the perforations or apertures in one of the two sheets may be aligned but out of registration with the perforations or apertures in the other one of the two sheets so that the tear paths do not leak. Using a tissue interface that comprises separation paths may simplify application of the tissue interface to a tissue site without tools.

FIG. 5C is a schematic view of another example of the tissue interface 120 in FIG. 5A, illustrating additional details that may be associated with some examples. In some embodiments, the standoffs 535 may be formed on both sides of at least one layer of the tissue interface 120, such as two hemispherical profiles, hemispherical cell 575 and hemispherical cell 585. The two hemispherical profiles may be aligned to form a generally spherical profile as shown in FIG. 5C. In some embodiments, when the first layer 515 and second layer 525 have substantially identical thickness or flexibility, the shape of the standoffs 535 may be substantially spherical as shown in FIG. 5C.

In alternative example embodiments, the standoffs 535 may have other profiles on both sides of at least one layer of the tissue interface 120. In alternative example embodiments, the standoffs 535 on both sides may not be aligned with each other. For example, the standoffs 535 on both sides of a tissue interface may overlap. In alternative example embodiments, the standoffs 535 may be formed on both sides by using sheets of polymeric films having a different thickness or flexibility. For example, the shape of the standoffs 535 on both sides may be asymmetric. For example, the standoffs 535 may have a width L2 of from 0.5 mm to 10 mm. In alternative example embodiments, the standoffs 535 may have a length D2 of from 0.5 to 10 mm.

FIG. 6 is a schematic drawing of an example of the therapy system 100 applied to a tissue site 605. In FIG. 6, the dressing 110 includes an alternative example embodiment of the tissue interface 120 of FIG. 5A. In some embodiments, the tissue interface 120 may be disposed at the tissue site 605 so that the second layer 525 is positioned facing the tissue site 605, and the hemispherical cells 575 can extend toward the tissue site 605. The hemispherical cells 575 have distal ends 610 that are adapted to contact the tissue site 605 when the tissue interface 120 is disposed at the tissue site 605. The standoffs 535 may also have side surfaces 615 that form a plurality of channels 620 for both negative pressure and instillation liquids. The hemispherical cell 585 portions of the standoffs 535 in FIG. 6 have distal surfaces 625 that can be adapted to contact the cover 125 when the cover 125 is placed over the tissue interface 120. The hemispherical cell 585 portions of the standoffs 535 in FIG. 6 also have side surfaces 630 that form a plurality of channels 635 between the cover 125 and the spacing region 505 for both negative pressure and instillation liquids. The apertures 570 extending through a region between adjacent standoffs 535 fluidly couple the channels 620 and the channels 635 so that the tissue interface 120 provides fluid communication from a fluid-delivery interface 640 to the tissue site 605 and from the tissue site 605 to a negative-pressure interface 645.

In other examples, a flat surface of the tissue interface 120 may be disposed against the tissue site 605. For example, the tissue interface 120 of FIG. 5B may be oriented so that the first layer 515 is applied to the tissue site 605, and the standoffs 535 support the cover 125 to create channels between the cover 125 and the space regions 505. The tissue interface 120 of FIG. 5B may also be inverted so that the standoffs 535 contact the tissue site 605.

In some embodiments, the dressing 110 may include at least one additional manifold. For example, a filler manifold may be disposed between the tissue interface 120 and the cover 125.

In operation, the negative pressure can be applied to the tissue interface 120 with or without the instillation of fluids. The tissue interface 120 may be configured to be compressed under the cover 125 at therapeutic levels of negative pressure, and may be configured to pass the negative pressure through the apertures 570 to the tissue site 605 to provide negative-pressure therapy as shown in FIG. 6. In alternative and additional embodiments, the negative pressure may be passed through borders or edges of the tissue interface 120 to the tissue site 605 to provide negative-pressure therapy. The standoffs 535 may be configured to release the polymer 565 to the tissue site 605 through the apertures 545, or alternatively or additionally through enzymatic degradation of the standoffs 535 by enzymes released from the tissue site 605. The polymer 565 may be biologically active and configured to provide substrates for matrix metalloproteinases (MMPs), nutrients or agents that reduces inflammation to the tissue site 605 in conductions with, before, or after the negative-pressure therapy. Alternatively or additionally, the instillation regulator 155 may provide instillation fluids comprising activating agents to the tissue site 605 to activate or enhance efficacy of the polymer 565 as substrates for MMPs, nutrients or agents that reduces inflammation.

The systems, apparatuses, and methods described herein may provide significant advantages. For example, the dressing 100 can be used for a duration of more than three days and up to seven days, 14 days or 21 days, and still be easy to remove. The dressing 100 may have alleviate pain associated with the dressing removal and may obviate the need for administration of pain medications.

Further, the dressing 100 can be a single, integral component that can manifold negative pressure and also deliver biocompatible or biologically-active polymers to a tissue site for an adjunctive therapy. In some examples, the polymers can be released at different rates to a tissue site to mimic a time-release treatment. The standoffs 535 may have varied sizes to store different amounts of the polymer.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A system for treating a tissue site, comprising:
a tissue interface comprising:
a film, and
a plurality of standoffs on the first film, wherein at least some of the standoffs enclose a composition comprising a biocompatible polymer;
a cover configured to be disposed adjacent to the tissue interface and to form a seal around the tissue site; and
a negative-pressure source fluidly coupled to the tissue interface through the cover.

2. The system of claim 1, further comprising a fluid control layer having a smooth surface adjacent to the plurality of standoffs.

3. The system of claim 1 or 2, wherein the tissue interface comprises apertures disposed between the standoffs and configured to allow fluid flow through the tissue interface.

4. The system of any of claims 1-3, wherein the standoffs are cells having a closed end.

5. The system of claim 4, wherein the cells have walls with a thickness of between 0.5 mm to 4.5 cm.

6. The system of claim 4 or 5, wherein at least some of the cells have a shape that is spherical, circular, polygonal or any combination thereof.

7. The system of any of claims 4-6, wherein the cells have a diameter of between 0.5 mm and 10 mm.

8. The system of any of claims 4-7, wherein the cells have a depth of between 0.5 mm and 10 mm.

9. The system of any of claims 4-8, wherein at least some of the cells have a spacing between two adjacent protrusions ranging from 2.0 mm to 10 mm apart.

10. The system of any of claims 4-9, wherein the cells are regularly spaced.

11. The system of any of claims 4-10, wherein the cells are irregularly spaced.

12. The system of any of claims 4-11, wherein at least some of the cells have perforations.

13. The system of claim 12, wherein the perforations comprise slits of between 0.5 to 2 mm in width or crosshatches.

14. The system of any of claims 1-13, wherein the tissue interface comprises thermoplastic elastomers, polyurethane, polyethylene, silicone, silicone-like materials, polyamide, polypropylene, polyethylene, polyvinyl chloride, ethylene vinyl acetate copolymers, polyvinyl alcohol, polyether block amide (PEBAX) polymers, or any combination thereof.

15. The system of any of claims 1-14, wherein the biocompatible polymer comprises a biologically-active polymer.

16. The system of any of claims 1-15, wherein the biocompatible polymer is biosorbable.

17. The system of any of claims 1-16, wherein the biocompatible polymer comprises anti-inflammatory, matrix metalloproteinases (MMP)-mitigating or antimicrobial polymers.

18. The system of any of claims 1-17, wherein the biocompatible polymer comprises collagen.

19. The system of any of claims 1-18, wherein the biocompatible polymer comprises oxidized regenerated cellulose.

20. The system of any of claims 1-19, wherein the biocompatible polymer comprises cross-linked collagen.

21. The system of any of claims 1-20, wherein the biocompatible comprises hyaluronic acid, chitosan, heparin, alginate, cellulose, fibrin, gelatin, chondroitin sulfate, agarose, dextran, carrageenan, silk, poly(ethylene glycol), poly(vinyl alcohol), polycaprolactone, polyphophazene, polyglycolic acid, rosin, lactose, sucrose, tapioca starch, and polyvinylpyrrolidone, or any combination thereof.

22. The system of any of claims 1-21, wherein the tissue interface is pliable.

23. The system of any of claims 1-22, wherein the tissue interface has a thickness of between 0.5 mm to 5 cm.

24. The system of any of claims 1-23, wherein the tissue interface has a thickness of between 0.5 mm to 1 cm.

25. The system of any of claims 1-24, wherein at least some of the standoffs form protrusions on one side of the tissue interface.

26. The system of any of claims 1-24, wherein at least some of the standoffs form protrusions on two sides of the tissue interface.

27. The system of claim 25 or 26, wherein the protrusions have walls with a thickness of between 0.5 mm to 4.5 cm.

28. The system of any of claims 25-27, wherein at least some of the protrusions have a shape that is spherical, circular, polygonal or any combination thereof.

29. The system of any of claims 25-28, wherein the protrusions have a width of between 0.5 mm and 10 mm.

30. The system of any of claims 25-29, wherein the protrusions have a length of between 0.5 mm and 10 mm.

31. The system of any of claims 25-30, wherein at least some of the protrusions have a spacing between two adjacent protrusions ranging from 2.0 mm to 10 mm apart.

32. The system of any of claims 25-31, wherein the protrusions are regularly spaced.

33. The system of any of claims 25-32, wherein the protrusions are irregularly spaced.

34. The system of any of claims 25-33, wherein at least some of the protrusions have perforations.

35. The system of claim 34, wherein the perforations comprise slits of between 0.5 to 2 mm in width or crosshatches.

36. The system of any of claims 1-35, wherein the biocompatible polymer is in a form of a liquid slurry.

37. The system of any of claims 1-35, wherein the biocompatible polymer is in a powder form.

38. A dressing, comprising:
a manifold comprising:
a film,
a first side and a second side,
a plurality of standoffs on at least the first side, wherein at least some of the standoffs enclose a composition comprising a biocompatible polymer; and
a cover configured to be adjacent to the second side of the manifold.

39. A manifold for treating a tissue site with negative pressure, the manifold comprising:
a polymer film; and
a plurality of standoffs enclosing a composition comprising a biocompatible polymer.

40. A method for treating a tissue site, comprising:
positioning a manifold adjacent to the tissue site, the manifold comprising a polymeric film having a plurality of cells, at least some of the cells enclosing a composition comprising a biocompatible polymer;
covering the manifold and the tissue site with a cover to form a sealed therapeutic environment; and
providing negative pressure from a negative-pressure source to the tissue site through the manifold.

41. The method of claim 40, wherein the cells are configured to not to block negative pressure to the tissue site.

42. The method of claim 40, wherein the cells are configured to release the biocompatible polymer over a time period of at least seven days.

43. The method of claim 40, wherein the cells are configured to release the biocompatible polymer over a time period of at least 14 days.

44. A method for manufacturing a manifold, comprising:
providing a polymeric film having a plurality of cells,
incorporating a composition comprising a biocompatible polymer into at least some of the cells; and
sealing the cells to encapsulate the composition.

45. The method of claim 44, wherein the composition comprise a liquid slurry.

46. The method of claim 45, further comprising freeze-drying the liquid composition.

47. The method of claim 44, wherein incorporating the composition into at least some of the cells comprises injecting the composition into at least some of the cells.

48. A method for manufacturing a manifold, comprising:
providing a polymeric film,
wrapping at least some portions of the polymeric film around a composition comprising a biocompatible polymer; and
sealing the portions of the polymeric film to create cells to encapsulate the composition.

49. The method of claim 48, wherein sealing the portions comprises sealing with heat.

50. The systems, apparatuses, and methods substantially as described herein.
